(19) United States
(12) Patent Application Publication
(10) Pub. No.: US 2009/0304812 A1
(43) Pub. Date: Dec. 10, 2009

(54) Topical Pharmaceutical Compositions

(76) Inventors: John Staniforth, Wiltshire (GB); Paul Goggin, Wiltshire (GB)

Correspondence Address: Davidson, Davidson & Kappel, LLC, 485 7th Avenue, 14th Floor, New York, NY, 10018, US

(21) Appl. No.: 12/086,046

(22) PCT Filed: Dec. 7, 2006
(86) PCT No.: PCT/GB2006/050435

§ 371 Date: May 14, 2009

(30) Foreign Application Priority Data
Dec. 7, 2005 (GB) 0524962.8

(51) Int. Cl. A61K 33/38; A61K 33/30; A61K 31/59; A61K 31/505; A61K 31/60; A61K 31/11; A61K 31/437; A61K 36/29; A61P 17/00; A61M 35/00

(52) U.S. Cl. 424/618; 514/557; 424/642; 514/167; 514/274; 514/159; 514/705; 514/293; 424/725; 604/309

(57) ABSTRACT

The present invention relates to compositions and applicator devices for providing accurate and localized administration of pharmaceutical compositions containing therapeutic agents to the skin. In particular, the invention relates to compositions which are solid at a temperature of about 250 C or less, and which soften upon continuous contact with the skin of a patient. The present invention allows a user to administer precise doses of a therapeutic agent by highly localized application of the composition to a desired skin region, without contacting surrounding skin regions, or the user's hand.

The present invention relates to pharmaceutical compositions for topical administration to the skin of a patient to provide a local therapeutic effect. The invention also relates to applicator devices for providing accurate and localized administration of such pharmaceutical compositions.

A wide variety of conditions and disorders may be treated with topically applied therapeutic agents providing a local therapeutic effect. In some cases, it is extremely important to ensure that the therapeutically active agent is applied exclusively to the skin area requiring treatment. In general, it is desirable to administer, a therapeutically active agent in the minimum amount necessary to produce the desired therapeutic effect. This is, of course, particularly true where the agent produces undesirable side effects, and some topically applied therapeutic agents are potentially harmful to healthy skin. In addition, where an accurate dose of a therapeutically active agent is required, application of the agent to healthy skin can result in the areas of skin that require treatment not receiving the necessary dose.

Unnecessary exposure of healthy skin to a therapeutically active agent can, for example, occur when the patient or caregiver uses his or her bare hand to apply the therapeutic composition to the affected area. Unnecessary exposure can also occur when the areas of the skin to be treated are particularly small or localised, increasing the likelihood that the therapeutically active agent is inadvertently applied to the surrounding healthy skin. For example, skin disorders such as acne can consist of isolated comedones, pimples etc. which are ideally individually treated with topical medication. This can be difficult, however, due to the small surface area to be treated and, as a result, surrounding healthy skin areas tend to be contacted by the medication.

Retinoids, such as isotretinoin or tretinoin, are used in the treatment of acne. Retinoids increase cell turnover of treated skin areas, allowing the top layer of skin to peel off. It may take up to 7 weeks for regular use of topical retinoids to have a noticeable effect and, as a result of their peeling action, the use of retinoids can actually result in an initial worsening of the condition of both healthy and affected skin. Retinoids are also known to cause side effects such as erythema, oedema and blistering. Temporary hyper- or hypopigmentation can occur with repeated application of topical tretinoin and increased susceptibility to sunlight has been reported. Limiting the area of the skin exposed to toxic drugs, such as retinoids, will reduce the undesirable side effects and can also improve patient compliance, as the worsening of symptoms associated with use of the drug may be avoided or is localized to just the affected areas which clearly need to be treated.

It is also important to limit exposure to drugs whose side effects may not be immediately apparent, but which have long-term consequences. Steroids, for example, interfere with collagen formation, and long term topical use can make skin thin and fragile. Blood vessels may also be affected by long-term topical steroid use, causing patients to bruise easily. Use of corticosteroids interrupts calcium/phosphate metabolism and, as a result, the risk of osteoporosis increases.

It is also important that precise doses of many topically applied therapeutic agents are administered. Administration of an exact dose can minimize undesirable side effects and allow the patient and/or the clinician to monitor the amounts of drug used and the effects of those amounts. As a result of such monitoring, adjustments can be usefully made to the doses and types of therapeutic agent administered, with the result of better attainment of optimum therapeutic levels.

However, at present therapeutic agents for topical administration are typically provided as creams, ointments, and gels etc., which are easy to spread onto and rub into the skin. Such creams, ointments, gels and the like are typically provided in a tube or a sachet. However, measuring out exact doses of such compositions can be difficult and this can result in the patient applying too much or too little of the therapeutic agent at each application. Applying too much of the therapeutic agent can result in significant and costly waste of the drug and may increase undesirable side effects. Applying too little of the therapeutic agent can result in the condition being inadequately treated. What is more, administration of inadequate doses of antibiotics is thought to have contributed to the widely acknowledged problem of antibiotic resistant bacteria.

A further problem often associated with known topically administered compositions is that many of the compositions comprise excipients which can cause allergic reactions in some subjects. For example, surfactants such as quaternary ammonium compounds have been reported as causing allergic reactions. Also, typically, conditions which are treated with topical compositions often manifest themselves as patches on the skin which are painful and/or itchy or as breaks in the skin. It is very important that a composition applied to such affected areas of skin does not contain irritants or the like. Ideally, the compositions should soothe the affected area.

Solutions to the problem of unnecessary exposure as a result of hand application include the patient or caregiver wearing a latex glove during application. However, many people find the feel of latex gloves unacceptable and uncomfortable, or they are allergic to latex. Latex gloves also add additional expense to drug therapy and may not be readily available to all patients. Further to this, the use of gloves does not provide a means of precise application of the composition to localized areas of affected skin, and therefore will not prevent unnecessary exposure to the drug through absorption by unaffected areas of skin.

Solutions to the problem of unnecessary exposure to therapeutic agents of healthy skin areas surrounding the affected area being treated tend to focus on means of locally applying the therapeutic agent, for example by use of an applicator. U.S. Pat. No. 5,681,574 describes an applicator incorporating liquid medication suitable for treatment of a relatively wide tissue area affected by acne. A disadvantage of such applicators, however, is that they frequently comprise medication that is in non-solid form. As such, the medication is easy to over-apply and is also liable to run, thereby coming in contact with the unaffected areas surrounding the affected area to which it is supposed to be applied.

In relation to the problems associated with providing exact doses of creams, ointments and gels etc., one solution has been for a patient to squeeze such a topical composition from a dispenser, such as a tube, along an index finger from the fingertip to the first joint and the amount of therapeutic agent thus dispensed is known as a fingertip unit (FTU). One FTU generally approximates to about 500 mg of a topical composition and is generally sufficient to cover an area that is twice that of a flat adult hand. Such administration is problematic in that the FTU is only an approximate measurement and its magnitude varies from patient to patient. Thus, use of the FTU does not achieve accurate dosing.

Thus, a means of topical administration of a pharmaceutical composition is required, wherein it is possible for a user to administer precise doses of a therapeutic agent by highly localized application of the composition to a desired skin region, without contacting surrounding skin regions, or the user's hand. In addition, it would be beneficial for such compositions to be free from allergens, irritants, etc.

According to a first aspect of the present invention, a pharmaceutical composition is provided for topical application to a mammalian patient, the composition comprising a therapeutic agent and a pharmaceutically acceptable carrier, wherein the composition is solid at a temperature of about 25° C. or less, and, upon continuous contact with the skin of a patient, softens to a consistency to effect substantial application of the therapeutic agent to a desired skin area of the mammalian patient within a time period of less than 10 minutes.

Compositions of the present invention should be stored at temperatures of about 25° C. or less, in accordance with storage conditions for most pharmaceutical compositions and compositions.

The compositions of the invention allow highly precise administration of the therapeutic agent, in terms of both the size of the dose administered and the area of skin to which the therapeutic agent is applied.

Preferably, a solid composition according to the present invention will soften when placed in continuous contact with the desired skin region to a consistency to effect application to the desired skin region within a time period of less than about 10, 5 or 2 minutes. The temperature at which a composition softens sufficiently to effect administration of the therapeutic agent is defined as its “softening point”.

The term “softening point” as used herein refers to a temperature at which a substantially solid dosage form starts to soften to a consistency that can be absorbed by the skin of a patient, so as to allow transdermal absorption of the therapeutic agent present in the composition.

The softening point of a substantially solid dosage form of a pharmaceutical composition according to the first aspect of the present invention can be determined visibly as the temperature at which the substantially solid dosage form starts to soften to a consistency that can be absorbed by the skin of a patient and as such can advantageously be substantially completely absorbed by the skin of the patient so as to leave little or no undesirable residue on the skin of a patient.

Alternatively, the softening point of a substantially solid dosage form of a pharmaceutical composition according to the first aspect of the present invention can be determined using a TA-XT2 texture analyser (Stable MicroSystems Ltd., UK), suitably equipped with a 5 kg load cell. The equipment is enclosed in a temperature-controlled chamber (capable of operating in the region of 60° C. to 200° C.). A tablet or other substantially solid dosage form according to the present invention may be enclosed in the chamber at the specified temperature for a time of at least 10 minutes. A 3 mm flat faced probe is pushed into the tablet or other substantially solid dosage form according to the present invention for a distance of 1 mm at a speed of 0.1 mm/sec. Measurements can be repeated at temperature increments of 1° C. and, at the temperature at which the peak force of resistance recorded (as measured by Texture Exceed software) falls to below 50% of that for a “solid” tablet or other substantially solid dosage form according to the present invention, the tablet or other dosage form is deemed to have “softened”.

Preferably, the softening point of a composition is the temperature at which, on heating the composition, its viscosity is reduced to 100,000 and preferably 50,000 centipoise or below.

The term “spreading point” as used herein refers to a temperature at which the composition has a spreading consistency. For example, the composition may flow under its own weight or at least can be spread upon the skin of a mammalian patient, for example, using finger pressure.

The mobility of a spreading composition may promote the absorption of the therapeutic agent into the skin by allowing movement of the therapeutic agent towards the skin, for example, by diffusion. The spreading point of a preparation may be measured using the TA-XT2 texture analyser mentioned above in relation to measurement of softening point and with this analyser the spreading point of a composition is the temperature at which outward flow of the composition is first observed on advance of the flat faced probe into the preparation.

Compositions in accordance with the present invention can have a softening point of not higher than the skin temperature of the patient to whom the composition is to be administered, preferably a living animal such as a human. Solid unit dosage forms in accordance with the invention can have an aspect ratio (wall:face) of less than 1:1.

The terms “therapeutic agent”, “active agent” or “pharmaceutically active agent” are used herein to denote any active substance having a therapeutic or prophylactic effect and which is suitable for topical administration to a patient, preferably a mammalian patient and most preferably a human patient. It is preferred that the therapeutic agent is suitable for topical application to the skin, and has a local effect. In some embodiments, the therapeutic agent exerts only a local effect upon topical application. In other embodiments, the therapeutic agent additionally has a systemic effect following topical application. Such agents include all of the drugs and classes of drugs referred to in following passages, as well as pharmaceutically acceptable equivalents or derivatives thereof, such as their pharmaceutically acceptable salts, esters, prodrugs and active metabolites. Isomers of all disclosed agents are also encompassed by this disclosure.

The term “local effect” as used herein relates to therapeutic effect that results from the provision of a composition containing one or more therapeutic agents for application to the skin of a patient, wherein the therapeutic agent has an effect on the area of skin to which it is applied, upon receptors in the skin, and/or upon receptors in the layers of the skin within close proximity to the site of application of the composition, and wherein the therapeutic agent is not administered to the bloodstream.

The term “systemic effect” as used herein relates to the therapeutic effect that results from administration of the therapeutic agent to the bloodstream.

Alternatively, the compositions of the present invention may be dispensed using a bespoke device which is capable of dispensing an accurate, predetermined amount of the composition.

According to some embodiments of the present invention, the therapeutic agent is an agent which is suitable for treating skin disorders such as acne, eczema, psoriasis, urticaria, contact dermatitis and the like. Alternatively, the therapeutic agent may be an agent which is known for use in topically applied anti-ageing treatments.

Where the compositions are for treating acne, the preferred active agents include: retinoids such as tretinoin, isotretinoin or retinoid related compounds such as adapalene; keratolyic agents, such as benzoyl peroxide, sulphur and any of a number of fruit acids and alpha-hydroxy acids; hormone modifiers; zinc; or antibiotics such as tetracycline, 4-epitetracycline, clindamycin, erythromycin and sulfonamides.

In some embodiments, pharmaceutical compositions according to the present invention do not include agents which are used for treating pain, inflammation, or for hormone-replacement therapy or contraception, or for administering local anaesthetics.

The pharmaceutical compositions according to the invention may comprise two or more therapeutic agents suitable for the treatment of acne, provided that they are compatible with one another under conditions of storage and use. One such combination is, for example, a combination of clindamycin and benzoyl peroxide.

The compositions may comprise a combination of one or more therapeutic agents suitable for the treatment of acne, combined with one or more other agents suitable for topical application, including: antimicrobial agents such as penicillins, cephalosporins, aminoglycosides, mupirocin, neomycin sulphate, polymyxins, silver sulfadiazine, azelaic acid, fusidic acid, or sodium hypochlorite; anti-inflammatory agents such as ibuprofen, aceclofenac, acemetacin, azapropazone, celecoxib, dexketoprofen, diclofenac sodium, diflunisal, etodolac, etoricoxib, fenbufen, fenoprofen, flurbiprofen, indometacin, ketoprofen, indometacin, medenamic acid, meloxicam, nabumetone, naproxen, piroxicam, rofecoxib, sulindac, tenoxicam, tiaprofenic acid, valdecoxib, salicylic acid and other salicylates; anti-viral agents such as acyclovir, penciclovir or idoxuridine; antiseptics such as triclosan (Irgasan DP 300), chlorhexidine, cetrimide, hexamine hippurate, cetylpyridinium chloride, dequalinium, phenolics such as trichlorophenol, chloroxylenol, povidone and iodine, phenoxy isopropanol, resorcinol, hexachlorophene, benzalkonium chloride; immunosupressants such as ciclosporin, methotrexate, pimecromilus or tacrolimus; anti-fungal agents such as imidazole and related compounds, clotrimazole, econazole, ketoconazole, miconazole, sulconazole nitrate, amorolfine, benzoic acid, nystatin, terbinadine, tioconazole or undecnoates such as methyl undecenoate or propyl undecenoate; other acne fighting compounds such as urea, allantoin, nicotinamide or hydroxyquinoline compounds; or skin-cleaning agents such as cationic surfactants and soaps, chlorine, astringents, oxidisers, dyes, hydrogen peroxide or potassium permanganate.

The pharmaceutical compositions may also include antipruritics such as crotamiton, calamine, doxepin hydrochloride or anti-irritants, such as alpha-bisabolol, farnesol, chamomile extract and glycyrrhetinic acid and/or solubilizers, such as glycerol, propylene glycol, polyalcohols, sorbitol and sorbitol derivatives, preferably sorbitan mono-oleate, solvents, antioxidants or moisturizers.

Where the compositions of the present invention are for treating eczema, they may include agents may be selected from the group of known eczema treatment agents. These agents include steroids such as hydrocortisone, clobetasone butyrate, betamethasone and betamethasone esters, hydrocortisone and hydrocortisone butyrate, clobetasol, clobetasol propionate and clobetasol butyrate, desonide, fludroxycortide, halcinonide, diflucortolone valerate, fluocinolone acetonide, triamcinolone acetonide, alclometasone dipropionate, beclomethasone dipropionate, desoximetasone, diflucorotolone valerate, fludroxycortide, fluocinolone and fluocinolone acetonide, flucinonide, fluticasone propionate, mometasone furoate, methylprednisolone aceponate, prednicarbate, desoximetasone, bethasone valerate, methylprednisolone aceponate.

The pharmaceutical compositions may comprise two or more therapeutic agents suitable for the treatment of eczema, provided that they are compatible with one another under conditions of storage and use.

The pharmaceutical compositions may comprise a combination of one or more therapeutic agents suitable for the treatment of eczema, combined with one or more of the following agents: anti-microbial, anti-fungal, antiseptic, antipruritic, anti-irritant, antibiotic, anti-viral, anti-inflammatory or skin-cleaning agents. Examples of such agents are provided above.

Alternatively, therapeutic agents which may advantageously be included in the compositions of the present invention include those which are usually administered topically for the treatment of psoriasis, for example vitamin D analogues such as deltanoids; methotrexate; or steroids.

The pharmaceutical compositions may comprise two or more therapeutic agents suitable for the treatment of psoriasis, provided that they are compatible with one another under conditions of storage and use.

The pharmaceutical compositions may comprise a combination of one or more therapeutic agents suitable for the treatment of psoriasis, combined with one or more of the following agents: anti-microbial, anti-fungal, antiseptic, antipruritic, anti-irritant, antibiotic, anti-viral, anti-inflammatory, immunosuppressants such as azathioprines or cyclosporins, or skin-cleaning agents as discussed above.

The compositions of the present invention are also suitable for the treatment of urticaria and contact dermatitis. Accordingly, the therapeutic agent could be selected from the group of antihistaminics, including doxepin, cetirizine, loratidine and fexofenadine, cimetidine, ranitidine, or leukotriene receptor antagonists, such as montelukast or zafirlukast, or corticosteroids such as hydrocortisone or desonide, mometasone furoate, methylprednisolone aceponate, prednicarbate, triamcinolone acetonide, fluocinonide, desoximetasone, bethasone valerate, methylprednisolone aceponate and mometasone furoate.

The pharmaceutical compositions may comprise two or more therapeutic agents suitable for the treatment of urticaria and contact dermatitis, provided that they are compatible with one another under conditions of storage and use.

The pharmaceutical compositions may comprise a combination of one or more therapeutic agents suitable for the treatment of urticaria or contact dermatitis, combined with one or more of the following agents: anti-microbial, anti-fungal, antiseptic, antipruritic, anti-irritant, antibiotic, anti-viral, anti-inflammatory, immunosuppressants such as azathioprines or cyclosporins, or skin-cleaning agents as discussed above.

The use of steroids in the treatment of eczema, psoriasis, urticaria and contact dermatitis is widely known. However, the side effects of long-term steroid treatment are also well recognized, including, as discussed above, the detrimental effects upon bone density and skin thickness. It is therefore recommended that sufferers of these conditions use a “ladder” or “step up-step down” approach to steroid use, wherein higher potency therapeutic agents, such as clobetasol propionate are applied to the affected skin areas when symptoms are most severe, followed by substitution with lower potency therapeutic agents, such as hydrocortisone, when the condition improves. The object of such an approach is to minimize exposure of the patient to drugs with known detrimental side effects. By providing a highly localized means of application of measured dose, the present invention provides controlled application of therapeutic agents and therefore aids achievement of this objective.

The present invention is also relevant to the treatment of skin cancer, including basal and squamous cell carcinomas. Accordingly, the therapeutic agent may be selected from the group of therapeutic agents used in the treatment of skin cancer, which include 5-fluoro uracil.

The pharmaceutical compositions of the present invention may comprise a combination of one or more therapeutic agents suitable for the treatment of skin cancer, combined with one or more of the following agents: anti-microbial, anti-fungal, antiseptic, antipruritic, anti-irritant, antibiotic, anti-viral, anti-inflammatory, immunosuppressant or skin-cleaning agents.

The present invention may be used for the topical application of anti-infection agents. Such agents have a wide variety of possible uses, for example, in the treatment of skin infections such as athlete's foot, manifestations of herpes simplex, impetigo, folliculitis, or ringworm. Therapeutic agents which may advantageously be included in pharmaceutical composition according to the present invention for the treatment of such infections include one or more of the following: anti-microbial, anti-fungal, antiseptic, antibiotic, anti-viral, anti-inflammatory, skin-cleaning agents or wart treatments such as salicylic acid, alkaylating agents formaldehyde, glutaraldehyde, silver nitrate, imiquimod or podophyllum.

The pharmaceutical compositions may comprise more than one anti-infection agent provided that they are compatible with one another under conditions of storage and use. The compositions may also include one or more other therapeutic agents.

In the treatment of some infections, such as warts or cold sores, it is sometimes recommended that the skin area to be treated is gently abraded during or prior to application of the therapeutic agent. Pharmaceutical composition comprising anti-infection agents according to the present invention may therefore further comprise an abrasive agent such as those that are commonly used in exfoliation creams. For example, the abrasive agent may comprise a mechanical abrasive such as microbeads or shell fragments, or an enzymatic abrasive, such as proteolytic enzymes.

The pharmaceutical compositions may comprise one or more agents known for use in topically applied anti-ageing treatments, such as humectants, suncreams, alpha or beta hydroxy acid, co-enzyme Q10, collagen, ceramides, hyaluronic acid, lanolin, parabens, squalene, vitamin C or vitamin E.

Preferably, the therapeutic agent or agents are present in the pharmaceutical compositions of the present invention in therapeutically effective concentrations. Preferably, the compositions include at least 0.01% by weight of active agent, based on the weight of the whole pharmaceutical composition.

The pharmaceutical compositions according to the present invention may comprise a means of substantially occluding the therapeutic agent from the air following application to the skin. The occlusive means is provided by the use of appropriate quantities of wax, oil or fat included in the carrier material of the composition.

Providing pharmaceutical compositions comprising a means of occlusion provides advantages for transdermal administration of the therapeutic agent. An important factor in the rate of absorption of a therapeutic agent through the skin is the hydration level of the skin. Application of an occlusive layer to the skin raises the temperature of the skin under the occlusive layer, causing dilatation of the pores of the skin and sweating, thereby hydrating the skin and enhancing absorption of agents that have been applied to the skin under the occlusive layer.

The pharmaceutically acceptable carriers included in the compositions of the present invention are preferably selected to allow the therapeutic agent to be carried in a stable manner. The carrier may have favourable organoleptic properties, for example, it may preferably have a non-oily feel upon application to the skin.

The carrier included in the compositions of the present invention is preferably substantially solid at a temperature of about 25° C. or less and softens to a consistency that allows for substantially complete absorption of the one or more therapeutic agents by the skin of the patient, so as to effect (preferably substantially complete) administration of the therapeutic agents to the patient, within a time period of less than about 10 minutes, preferably less than about 5 minutes, more preferably less than about 3 minutes and most preferably less than about 1 minute following application to the area of skin.

Typically, it is preferred that the carrier medium included in the substantially solid dosage form of the present invention may soften, and advantageously may be converted to a spreading consistency, at a temperature in the range of 30 to 37° C.

In its substantially solid form, the composition of the invention preferably has a size and shape suitable for application to a selected area of skin.

More particularly, it is preferred that the shape and configuration of the substantially solid dosage form is determined by the softening point of the composition and/or the carrier medium. It may be preferred that a substantially solid dosage form according to the present invention comprises a substantially unitary form; alternatively, it may comprise a plurality of discrete particles (such as a plurality of granules or the like) that can be absorbed by the skin of a mammalian patient. Preferably, the plurality of substantially discrete particles is provided in a sealed member (such as a capsule, sachet, blister package or the like) from which they are dispensed and applied to the skin of a patient.

Any component commonly used for suppositories can be used as carriers in the compositions of the present invention which soften upon application to the skin. These components include those derived from mammalian, vegetable or mineral origins, and materials partially or totally synthesized. Specific examples of such carriers include oils and fats of mammalians or vegetable origin, such as olive oil, corn oil, castor oil, cottonseed oil, wheat germ oil, cacao butter, hydrogenated oils, etc.; hydrocarbons, such as squalane, petrolatum, solid paraffin, liquid paraffin, etc.; and waxes, such as jojoba oil, carnauba wax, bees wax, lanolin, etc. Examples of partially or totally synthesized fatty acid esters include glycerol, mono-, di-, or triglycerides of medium or higher fatty acid, such as saturated linear fatty acid, for example lauric acid, myristic acid, palmitic acid, stearic acid, etc., or unsaturated linear fatty acids, for example oleic acid, linoleic acid, linolenic acid, etc. Commercially available carriers which are suitable include Witepsol (manufactured by Dynamit Nobel), Pharmasol (manufactured by Nippon Oil and Fats Co.), Isocacao (manufactured by Kao Corp.), SB (manufactured by Taiyo Oil and Fats Co.), Novata (manufactured by Henkel), Suppocire (manufactured by Gattefosse Co.), and the like. Examples of other synthetic products include polyethylene glycol, for example, macrogole, setomacrogole, etc., as well as derivatives thereof, for example, setomacrogol.

In order to obtain the desired softening point of the compositions of the present invention, different carriers can, if necessary, be combined in order to increase or decrease the softening point to obtain a suitable product. For example, in order to decrease the softening point, a plasticizer can be added, e.g., glyceryl monostearate, myristyl alcohol, polysorbate 80, propylene glycol or combinations thereof. In order to increase the softening point, a hardener can be added, e.g., beeswax, cetyl alcohol, stearic acid, stearyl alcohol, aluminium monostearate, aluminium distearate, aluminium tristearate, bentonite, magnesium stearate, colloidal silicon dioxide or combinations thereof.

A carrier for use according to the present invention may comprise any ingredient suitable for use in a pharmaceutical composition and possessing the desired properties for enabling topical administration of a dose of at least one therapeutic agent, provided that it is suitable for topical application and transdermal administration. For example, the carrier may include a cellulose or one or more ingredients selected from the group consisting of ingredients of the type suitable for use in suppositories including, for example, one or more glycerides (such as, for example, one or more glycerol esters of saturated fatty acids or one or more polyglycolysed glycerides, cocoa butter, theobroma or the like), one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like), and any of the preceding ingredients can be optionally mixed with one or more organic oils (including, for example hydrogenated vegetable oils) or the like.

It is often preferred that a carrier employed in a pharmaceutical composition according to the present invention comprises, and more preferably consists essentially of, one or more glycerides, including, in particular, one or more glycerol esters of C8-C18 fatty acids or one or more polyglycolysed glycerides.

Suitably, the carrier of a pharmaceutical composition according to the present invention comprises, or consists essentially of, a mixture of glycerides, where the glycerides can be one or more mono-, di- or tri-glycerides, optionally wherein the glycerides comprise glycerol esters of C12-C18 fatty acids. In one embodiment, the glyceride mixture is a Witepsol grade product. More particularly, the carrier may comprise, or consist essentially of, a Witepsol grade product available under any of the trade marks Witepsol H5, Witepsol H15, Witepsol H32, Witepsol S51, Witepsol S55, Witepsol S58, Witepsol W25 and Witepsol W32. In a particularly preferred embodiment, the pharmaceutical compositions according to the present invention include carriers which are Witepsol grade products available under any of the following trade marks Witepsol H5, Witepsol H15, Witepsol S51 and Witepsol S55. The Witepsol grade product available under the trade mark Witepsol H15 is particularly suitable.

In a particular embodiment of the present invention, the carrier employed in the compositions consists essentially of a Witepsol grade product substantially as described above.

Alternatively, the carrier comprises, or consists essentially of, a mixture of glycerides, where the glycerides can be selected from the group consisting of mono-, di- and tri-glycerides, the glycerides comprising glycerol esters of Cg-Cie fatty acids or one or more polyglycolysed glycerides. In one embodiment, glyceride mixtures available under the trade marks Gelucire or Suppocire are used, such as any of the following: Gelucire 33/01, Gelucire 39/01, Gelucire 43/01, Gelucire 44/14, or any of the Suppocire Standard type, Suppocire N type or Suppocire P type products.

Alternatively, the carrier used in a pharmaceutical composition according to the present invention comprises, or consists essentially of, cocoa butter.

Pharmaceutical compositions according to the present invention may further comprise, where appropriate, additional ingredients such as one or more penetration enhancers (which may be surfactants, alcohols, esters, glycols or the like or any other suitable penetration enhancer), humectants, surfactants (which may be cationic, non-ionic, anionic or polymeric), emulsifiers, antioxidants, preservatives, clays, antifoaming agents, spreading agents, emollients, barriers, solubilising agents for the therapeutic agent and the like.

Pharmaceutical compositions according to the present invention may also comprise solvents, such as ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

The presence of solvents in compositions according to the present invention aids administration of the therapeutic agent. The extent to which, and speed with which administration of a therapeutic agent from a topically applied composition occurs is associated with the depth and rate of penetration of the therapeutic agent into and through the skin. The presence of solvents in compositions according to the present invention aids solubilization of the drug within the composition. Solvents for use in the present invention are also chosen in accordance with their ability to cross or bridge the layers of the skin, and in particular the tight junctions between the corneocytes located within the stratum corneum. The presence of solvents in the present invention thus enhances the rate of transdermal absorption, and the depth of penetration of the therapeutic agent by solubilizing the agent, and effecting diffusion of the agent through the skin.

Pharmaceutical compositions according to the present invention may further comprise organoleptic agents to improve the organoleptic properties of the composition. Such agents include almond oil, glycerol, linseed oil, monoethanolamine oleate, grape oil, mace oil, isopropyl myristate, isopropyl palmitate, palm kernel oil, theobroma oil, and wool alcohols. The inclusion of organoleptic agents can be used, for example, to enhance the feel of the composition, which can improve patient compliance. In addition, such agents can have a perceived cooling effect, which can provide a positive psychological effect, particularly when the composition is used on inflamed or reddened skin.

Pharmaceutical compositions according to the present invention may further comprise sensory cues, such as anise oil, citronella oil, clove oil, eucalyptol, eucalyptus oil, eugenol, juniper oil, lemon grass oil, lemon oil, tepeneless lemon oil, melaleuca oil, neroli oil, nutmeg oil, olive oil, orange oil, terpeneless orange oil, poppy seed oil, pine oil, rose oil, sage oil, spearmint oil, lavender oil, thyme oil, vanillin.

The inclusion of such cues in the composition can provide the patient with pleasant sensory feedback upon use, allows the patient and/or person applying the formulation to recognize that administration has occurred, and may aid recollection of administration. Such factors can improve patient compliance and provide a positive psychological effect.

Pharmaceutical compositions according to the present invention may further comprise insect repellents such as citronella or lemon grass.

In some embodiments of the present invention, the compositions are substantially free of penetration enhancers. In such embodiments, the compositions are preferably prepared using a process carried out under aseptic conditions.

The use of preservatives can be undesirable, as they may provoke allergic reactions in susceptible patients, and the present invention may be advantageous in avoiding or reducing the risk of such allergic reactions. Preservatives that have been associated with allergic reactions include chlorocresol, hydroxybenzoates (parabens), polysorbates, sorbic acid and the like, and these preservatives are included in a large number of known topical compositions, including, for example, compositions available under any of the following trade marks: Drapolene, Medicaid, Siopel, Sprilon, Eurax, Efcortelan, Mildison, Fucidin H, Nystaform, Quinocort, Terra-Cortril Nystatin, Timodine, Locoid, Locoid Crelo, Modrasone, Propaderm, Betnovate, Betnovate RD, Diprosone, Dermovate, Eumovate, Trimovate, Nerisone, Haelan, Synalar, Ultralanum Plain, Zorac, Carbo-Dome, Exorex, Differin and Exelderm.

In a particularly preferred embodiment of the present invention, the pharmaceutical compositions are substantially free of the types of preservative generally included in compositions intended for dermal or transdermal administration, or at least they include such preservatives in amounts that are less than those generally required in compositions intended for dermal or transdermal administration, or they include such preservatives in amounts that generally do not provoke substantial allergic reactions in susceptible patients, substantially as hereinafter described.

The preservatives generally employed in compositions intended for dermal or transdermal administration are included to prevent or reduce contamination of such compositions. Contamination is a particular problem where a composition is repeatedly exposed to the atmosphere or is repeatedly handled. Preservatives may not be required in compositions of the present invention where the compositions are in the form of unit doses, especially if these doses are individually packaged.

Pharmaceutical compositions according to the present invention may, however, comprise one or more preservatives, such as phenoxyethanol or the like, that are included typically to substantially prevent contamination of the compositions according to the present invention during manufacture but are not generally of the type employed to prevent infection due to manual application as hereinbefore described.

In further embodiments of the present invention, the compositions are substantially free of antioxidants. In such cases, it is preferred that the compositions are packaged in a substantially inert atmosphere, such as nitrogen or the like.

The use of antioxidants can provoke allergic reactions in susceptible patients and the present invention may be advantageous in avoiding or reducing the risk of such allergic reactions in susceptible patients. Antioxidants that have been associated with allergic reactions include butylated hydroxyanisole, butylated hydroxytoluene and the like, and are known to be available in prior art topical compositions, such as those compositions available under any of the trade marks Imuderm, Siopel and the like.

In a particularly preferred embodiment of the present invention, the pharmaceutical compositions are substantially free of antioxidants of the type generally included in compositions for dermal or transdermal administration, or at least they include such antioxidants in amounts less than generally required in compositions intended for dermal or transdermal administration, or at least they include such antioxidants in amounts that generally do not provoke substantial allergic reactions in susceptible patients substantially as hereinafter described.

Antioxidants are generally employed in compositions intended for dermal or transdermal administration in order to prevent the fats present in such compositions becoming rancid and to prevent oxidation of the composition following opening of the packaging within which the composition is kept. Antioxidants may not be required in compositions of the present invention where the compositions are in the form of unit doses, especially if these doses are individually packaged.

Methods of preparing the softening compositions referred to above are disclosed in WO 02/00203 A1, the entire disclosure of which is hereby incorporated by reference.

In certain embodiments of the invention, the compositions are in the form of unit dosage forms. This means that an exact dose of the therapeutic agent can be provided, which in turn helps to ensure that an accurate, predetermined dose of the therapeutic agent is actually administered to the patient. Such unit dosage forms may be packaged individually, for example in containers such as tubes or sachets, or as a conventional blister pack.

Packaging the pharmaceutical compositions of the invention in this manner increases shelf life and prevents the pharmaceutical composition from becoming prematurely oxidised or degraded. Packaging can be carried out in a nitrogen atmosphere to provide further resistance to degradation on storage.

The compositions according to the present invention may be provided as at least one separately packaged unit dosage form. In one particularly preferred embodiment, the unit dosage form is a tablet. The unit dosage forms may be individually contained in a plastic container having a removable or breakable enclosure for dispensing each unit dose.

The pharmaceutical compositions of the present invention are preferably solid during manufacture.

In certain embodiments of the present invention the pharmaceutical composition for topical administration is a compacted granulate comprising one or more doses of one or more therapeutic agents and a pharmaceutically acceptable carrier, said compacted granulate having a softening point of not higher than skin temperature of a mammalian patient.

In certain embodiments, the composition has a shape to facilitate the topical application. For example, the composition can have: at least one flat surface; at least one concave surface; at least one convex surface; two flat surfaces; two concave surfaces; or two convex surfaces. The composition may be in the form of a standard tablet, spherical or half-spherical. Bullet shaped and conical shaped compositions are not preferred in the present invention.

Preferably the composition is shaped so that it has a surface area which is suitable for contact with a small area of the skin, for example an area of skin not more than 400, 300, 250, 200, 150, 100, 50, 25, 20, 19, 18, 17, 16, 15, 14, 13, 12, 11, 10, 9, 8, 7, 6, 5, 4, 3, 2, 1, or 0.5 mm².

In some embodiments of the invention, the compositions are provided in the form of a unit dosage form. The unit dosage forms are preferably substantially solid when stored under conditions normal for pharmaceutical formulations and compositions.

In preferred embodiments, the unit dosage forms have a total weight of from about 50 mg to about 1 g, preferably from about 100 mg to about 900 mg and more preferably from about 250 mg to about 750 mg. That said, unit dosage forms according to the invention can weigh in excess of 1 gram, if desired.

Solid unit dosage forms in accordance with the invention can be prepared by a tableting process. Typically, tableting involves introducing a flowable composition, such as a mixture of at least one therapeutic agent and a carrier, into a tableting press and compressing the mixture to yield a substantially solid form, typically a tablet.

A process for preparing a pharmaceutical composition suitable for use in or with embodiments of the present invention can comprise cooling at least a portion of a mixture of at least one therapeutic agent and pharmaceutically acceptable carrier, wherein the cooling can improve handling properties of the mixture and may also increase the speed of tableting. The cooling step may be carried out prior to and/or during the shaping of the mixture into the dosage form. Preferably, the mixture is cooled to a temperature of not more than about 15° C., advantageously not more than about 10° C. and, for example, not more than about 0° C., prior to and/or during shaping.

The cooling may be effected at least in part by using a cooled tableting press. Additionally or alternatively, the mixture may be cooled prior to being introduced into the tableting press.

The carrier preferably allows the mixture to be shaped into a substantially solid dosage form at temperatures of up to about ambient or room temperature (e.g. at temperatures of up to 20, 21, 23, 24, 25, 26, 27, 28, 29, or 30° C.).

In accordance with a second aspect of the present invention, there is provided an applicator for topically applying to the skin or other exterior region of a human or animal body, a pharmaceutical composition according to the first aspect of the present invention, said applicator comprising a receiving means for receiving and carrying the pharmaceutical composition so that the composition may be applied directly to the skin, and a grip for enabling a user to hold and manipulate the applicator. The composition may be in the form of a unit dosage form.

In certain embodiments, the receiving means of the applicator provides an exposed surface area of the composition for contact with the patient of not more than 400, 300, 250, 200, 150, 100, 50, 25, 20, 19, 18, 17, 16, 15, 14, 13, 12, 11, 10, 9, 8, 7, 6, 5, 4, 3, 2, 1, or 0.5 mm².

Preferably, such applicators are provided in individual, sealed packaging.

In certain embodiments, the receiving means is arranged to define a recess that can accommodate the pharmaceutical composition in its substantially solid form, optionally in the form of a unit dosage form, wherein the depth of the recess can be adjusted automatically or manually to allow a surface of the composition accommodated in the recess to be exposed for application to the skin and to allow substantially all of the composition to be applied through skin contact induced erosion at said exposed face of the composition. Further to this, in certain embodiments, the applicator further comprises an actuator, actuation of which adjusts the depth of the recess in order to control the exposure of the composition.

Preferably, the exposed face of the pharmaceutical composition held within the recess of the applicator has a surface area of not more than 400, 300, 250, 200, 150, 100, 50, 25, 20, 19, 18, 17, 16, 15, 14, 13, 12, 11, 10, 9, 8, 7, 6, 5, 4, 3, 2, 1, or 0.5 mm².

Preferably, the recess is configured to confine at least an uneroded portion of the composition and thus to provide resistance to the bodily displacement of the uneroded portion from the recess. More preferably, the recess is configured to resist displacement of the uneroded portion of the composition from the recess other than as a result of skin contact induced erosion at said exposed face of the composition.

In certain embodiments, the applicator includes a valve disposed at an opening of the receiving means wherein upon actuation, the valve is movable between an open position to allow exposure of the substantially solid composition through the opening and a closed position to seal the opening. In certain aspects, actuation of the actuator may cause a positive pressure, which can cause the valve to move from a closed position to an open position.

The actuator hereinbefore described is preferably positioned on the applicator to allow a user to actuate and then apply the composition to the skin with one hand. In preferred embodiments, the user does not have to reposition the hand from an actuation position to an application position and all steps of actuation and application of the composition to the skin of a patient can be performed with minimal or no repositioning of the hand.

In certain embodiments, the actuator may comprise a button, and the actuator may be flush with the surface of the applicator or can be recessed, to minimize the accidental actuation during the application process. In certain embodiments, the button can be moved between a non-actuated position and an actuated position. Further to this, certain embodiments may comprise a button spring mechanism, which causes the button to return to the non-actuated position after actuation.

When the actuation mechanism includes a lead screw, then the lead screw may preferably include a mechanism, such as a ratchet, adapted to reduce the back pressure in the container.

An actuator useful for the applicator described in the present invention can also comprise other types of mechanisms for exposing the face of a composition from the receiving means. For example, the actuator can comprise a button, a rack, a pinion and a lead screw in operative connection with each other. A spring mechanism can also be used.

In another embodiment, the receiving means of the applicator comprises a surface adapted to retain the composition, optionally a unit dosage form, and the composition may be carried on or coupled to the receiving means.

The substantially solid composition can be attached to the receiving means of the applicator by mechanical, physical or chemical means. Attachment can be achieved by the application of pressure, heat or an adhesive agent, depending upon the characteristics of the composition. Adhesives suitable for fixing solid compositions, preferably solid dosage forms, in place include polydimethylsiloxane, collodion, cyanoacrylates and polymers of acrylic acid, including polyacrylamides and polymethacrylates.

In certain embodiments, the surface of the receiving means may be roughened, or could include one or more raised portions capable of penetrating at least part-way into a substantially solid composition.

In alternative embodiments, the composition may be coupled to the applicator by heating and melting or softening a surface of the composition, contacting that surface with the receiving means and cooling the resulting assembly to harden the melted or softened portion of the composition and cause it to become bonded to the receiving means. This is especially useful when the receiving means includes one or more raised portions, as it facilitates the penetration of such a portion or portions into the composition.

In alternative embodiments, the applicator may also include an intermediate member attached to the composition, and the receiving means of the applicator may be configured to be removably attachable to the intermediate member.

In certain embodiments, the grip of the applicator is configured to be held by a user, and may be held between a finger and thumb, to enable the user to move the applicator so as to apply the composition to desired skin regions. The applicator in the present invention may further comprise a shroud around at least a portion of the grip, or a zone in the vicinity of the grip, wherein the shroud is arranged to shield said zone or portion of the grip from the pharmaceutical composition such that a user holding the applicator by the grip is protected from inadvertent contact with the composition.

Embodiments of the invention are now further described by way of example only, with reference to the accompanying drawings, in which:

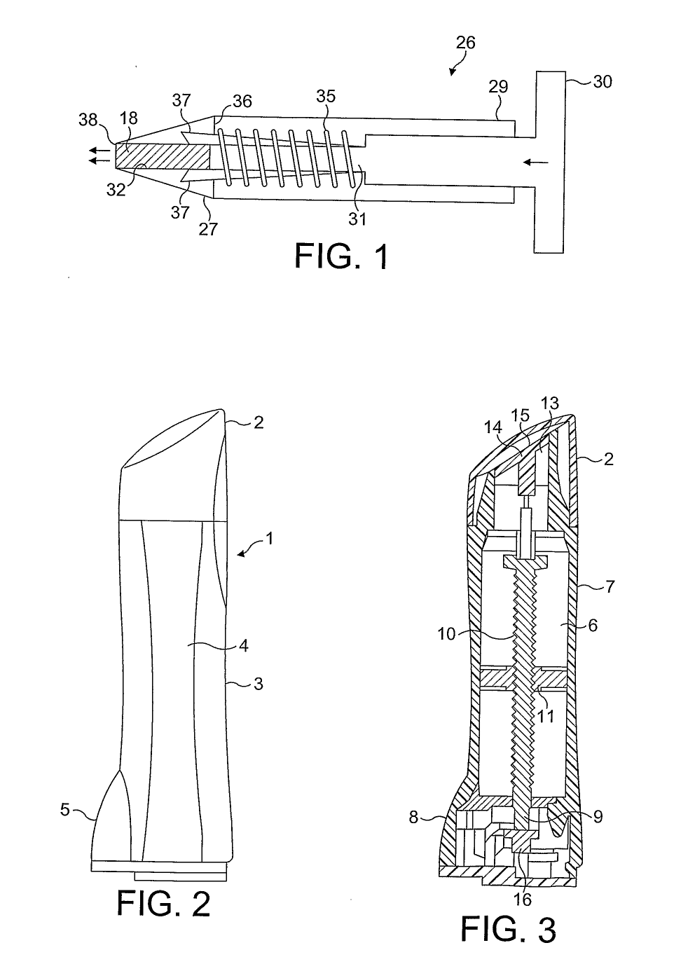

FIG. 1 shows a cross-sectional view of an applicator in accordance with an embodiment of a second aspect of the present invention;

FIG. 2 is a view of an applicator in accordance with an alternative embodiment of the second aspect of the present invention;

FIG. 3 shows a cross-sectional view of the embodiment shown in FIG. 2;

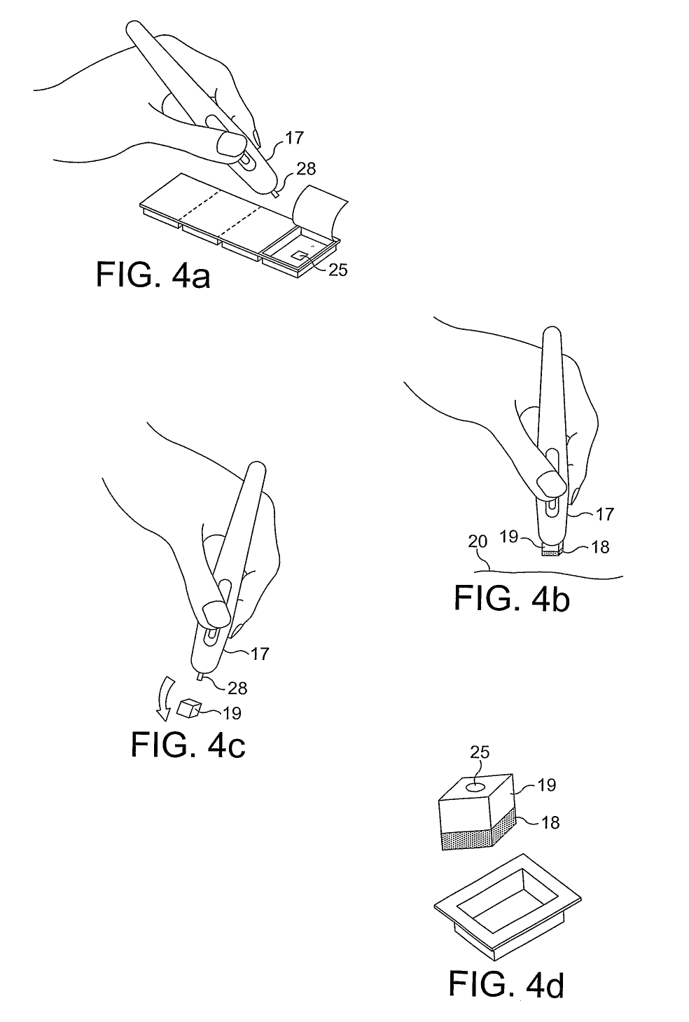

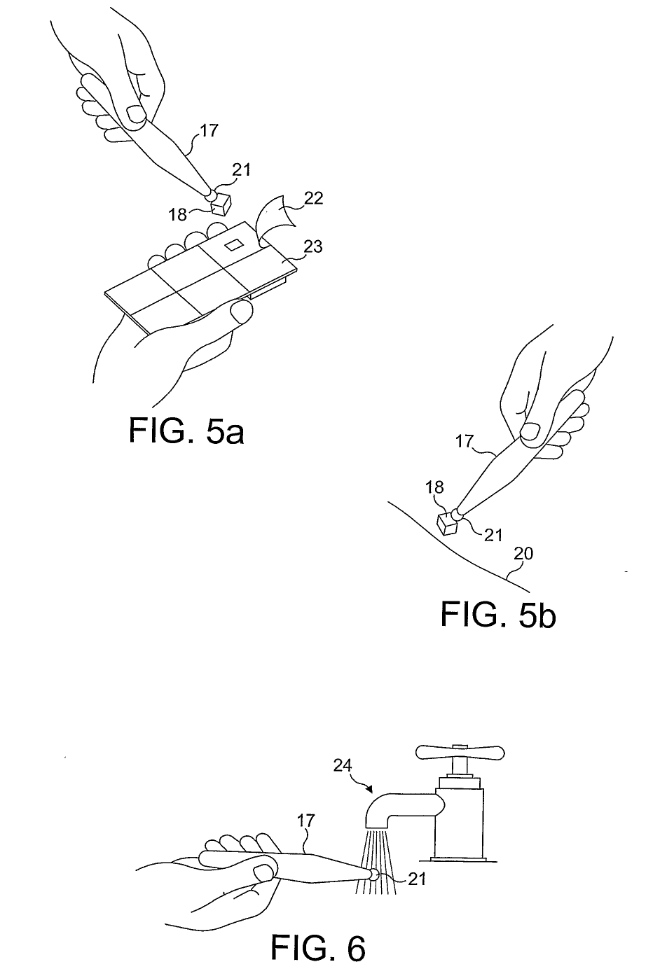

FIGS. 4a, b, c and d, FIGS. 5a and b and FIG. 6 show a view of an applicator in accordance with an alternative embodiment of the second aspect of the present invention.

FIG. 1 shows an applicator 26 with a receiving means 27, grip 29, and actuator 30, wherein the actuator has been partly actuated in accordance with the description below. The receiving means 27 comprises a recess 32. The receiving means further comprises two flanges 37, which are capable of closing around the sides of a unit dose. Unit or measured doses of the pharmaceutical composition 18 may be inserted into the recess in the receiving means. A spring 35 is disposed between a shoulder in the grip 36 and the end of the actuator 30. Actuation of the actuator 30 causes compression of the spring 35 which causes opening of the flanges 37 which allows the unit dose to move through the receiving means 27 and the opening 38 thereby exposing at least a face of the unit dose for application to the skin. Release of the actuator 30 causes the flanges to close around the unit dose, thereby holding it in place.

FIG. 2 shows the exterior of an alternative embodiment of an applicator 1 with a receiving means 2. The grip 3 may include side portions 4 to provide an attachment to the grip 3 also in an opened position, which will be explained further below. The grip 3 may have a generally cylindrical shape and the applicator 1 may be sized so as to be hand-held. Button 5 may be disposed at one end of the grip 3.

As shown in FIG. 3, a pharmaceutical composition 6 which may include one or more therapeutic agents and a carrier, is held within a cylindrical grip 7. Button 5 may be rigidly connected to rack 8 which is in operative connection with pinion 9 so that a displacement of rack 8 in its longitudinal direction causes pinion 9 to rotate. The pinion 9 may be rigidly connected to lead screw 10 which may be disposed longitudinally within the grip 7. The lead screw 10 in turn may be operatively connected to a piston 11 in such a way so that a rotation of the lead screw 10 causes a displacement of the piston 11 in a longitudinal direction of the lead screw 10.

The piston 11 may extend from the lead screw 10 at its centre, to an inner wall of the grip 7 at its outer perimeter. Preferably a seal is formed between piston 11 and both the lead screw 10 and the grip 7 so that upon actuation of button 5 the rack 8 causes the pinion 9 and lead screw 10 to rotate, thus causing the piston 11 to move incrementally in an upward direction pushing the pharmaceutical composition 6 upward with it into and through the receiving means 2 and through opening 13. An outlet valve 14 covers outlet opening 13 in its closed position.

The lead screw 10 at its distal end from the receiving means, may be operatively connected to a mechanism 16. Mechanism 16 may be configured to cause the lead screw 10 to displace in a longitudinally upward direction as button 5 is depressed, and to drop back down again to its original longitudinal position when button 5 reaches its fully depressed position or when button 5 travels back to its original (not actuated) position.

As shown in FIGS. 4a, b, c, and d, the applicator 17 may be pen-shaped. The applicator is configured to be removably attachable via a receiving means 28, either directly to a unit dose 18 or to an intermediate member 19 that is directly attached to the unit dose 18. Where the receiving means is to be attached to an intermediate member, the receiving means may comprise, for example, a pin which is inserted into a fitting hole 25 in the intermediate member 19. The user should hold the intermediate member 19 using the applicator instrument 17 for applying unit dose 18 onto the desired skin region 20. After the unit dose 18 has been applied to the desired skin region, the user may dispose of intermediate member 19 (and any unused portion of unit dose 18) from the applicator, 17, such as by activating a lever on applicator, 17.

As shown in FIGS. 5a, b and 6, the applicator 17 may include an intermediate member 21 connected to the applicator to form a one-piece instrument. The user peels back a protective layer 22 from a blister pack 23 which contains unit doses of the composition. The applicator 17 is held by the user and the intermediate member 21 is pressed onto a unit dose 18 within the blister pack. The intermediate member 21 adheres to the unit dose 18 and the user applies the unit dose 18 onto the desired skin region 20 as shown in FIG. 5b. After the unit dose has been applied to the desired skin region 20 the user washes any excess pharmaceutical composition from the intermediate member 21 and applicator instrument 17 for example by holding it under a running faucet 24.

According to a third aspect of the present invention, a kit is provided comprising a pharmaceutical composition according to the first aspect of the present invention and an applicator. Preferably, the applicator is one according to the second aspect of the present invention.

In one embodiment, the kit comprises at least one dose of the pharmaceutical composition and at least one applicator according to the second aspect of the present invention. Kits in accordance with the invention can include instructions for coupling the composition to the applicator for use.

In a fourth aspect of the present invention, there is provided a method of treating an animal or human body comprising topically applying a pharmaceutical composition according to the first aspect of the present invention.

In certain embodiments, the method involves the use of an applicator according to the second aspect of the present invention, involving holding the grip of the applicator and contacting the face of the composition with the skin region to be treated, so as to apply said composition to said skin region.

According to a fifth aspect of the present invention, there is provided a pharmaceutical composition according to the first aspect of the present invention, for treating: acne, eczema, psoriasis, urticaria, contact dermatitis, warts or cold sores or other skin disorders of a patient by topically applying the composition.

There is also provided the use of a composition according to the first aspect of the present invention in the manufacture of a medicament for topical application to the skin of a patient to treat acne, eczema, psoriasis, urticaria, contact dermatitis, warts or cold sores or other skin disorders. Preferably, the patient is mammalian.

The compositions of the present invention can also be used in combination with an oral therapy. The highly localized topical application of part of a dose of a therapeutic agent may enable a much smaller than usual oral dose to be effective. Such a system of dual or serial administration has many advantages, including ease of application of the subsequent doses, and accurate monitoring of both the amount of therapeutic agent administered and the effects of the amounts given. This would result in improved attainment of therapeutic effects whilst reducing the resultant toxicity and side effects.

The present invention will now be illustrated by the following Examples, which do not limit the invention in any way.

### EXAMPLE 1

| Ingredients: | % w/w |
|---|---|
| Softisan 133 | 83 |
| Dry Flo AF Pure | 9 |
| Migylol 812N | 5 |
| Fitoderm (veg squalene) | 1 |
| Mupirocin calcium | 2 |

### Method of Preparation for Example 1

All ingredients excluding the mupirocin calcium were melted down until molten, and the temperature of the bulk was then maintained at 60° C. The mupirocin calcium was carefully sheared into the bulk using a Silverson mixer. The bulk was solidified by exposing to low temperature, for example, 4° C. The solidified bulk was milled down and granulated also at low temperature, for example, 4° C.

### EXAMPLE 2

| Ingredients: | % w/w |
|---|---|
| Softisan 133 | 82 |
| Dry Flo AF Pure | 8 |
| Migylol 812N | 5 |
| Isopropyl Myristate | 2.5 |
| Fitoderm (veg squalene) | 1.5 |
| Beclomethsone Dipropionate | 1 |

### Method of Preparation for Example 2

All ingredients excluding the beclomethasone dipropionate and dry flo were melted down until molten, and the temperature of the bulk was then maintained at 60° C. The beclomethasone dipropionate and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 3

| Ingredients: | % w/w |
|---|---|
| Softisan 133 | 83 |
| Dry Flo AF Pure | 5 |
| Migylol 812N | 2 |
| Salicylic acid | 10 |

### Method of Preparation for Example 3

All ingredients excluding the salicylic acid and dry flo were melted down until molten, and the temperature of the bulk was then maintained at 60° C. The salicylic acid and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 4

| Ingredients: | % w/w |
|---|---|
| Softisan 133 | 82.5 |
| Dry Flo AF Pure | 8 |
| Migylol 812N | 5 |
| Isopropyl Myristate | 2.5 |
| Fitoderm (veg squalene) | 1.5 |
| Podophyllum | 0.5 |

### Method of Preparation for Example 4

All ingredients excluding the podophyllum and dry flo were melted down until molten, and the temperature of the bulk was then maintained at 60° C. The podophyllum and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 5

| Ingredients: | % w/w |
|---|---|
| Softisan 133 | 82.9-82.5 |
| Dry Flo AF Pure | 8 |
| Migylol 812N | 5 |
| Isopropyl Myristate | 2.5 |
| Fitoderm (veg squalene) | 1.5 |
| Calcipitriol | 0.1-0.5 |

### Method of Preparation for Example 5

All ingredients excluding the calcipitriol and dry flo were melted down until molten, and the temperature of the bulk was then maintained at 60° C. The calcipitriol and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 6

| Ingredients: | % w/w |
|---|---|
| Softisan 133 | 83 |
| Dry Flo AF Pure | 10 |
| Migylol 812N | 3.5 |
| Fitoderm (veg squalene) | 1.5 |
| Isotretinoin | 1 |
| Hydrocortisone | 1 |

### Method of Preparation for Example 6

All ingredients excluding the isotretinoin, hyrdocortisone and dry flo were melted down until molten, and the temperature of the bulk was then maintained at 60° C. The isotretinoin, hyrdocortisone and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 7

| Ingredients: | % w/w |
|---|---|
| Softisan 133 | 83 |
| Dry Flo AF Pure | 10 |
| Migylol 812N | 4 |
| Fitoderm (veg squalene) | 1 |
| Hydrocortisone | 1 |
| Fusidic acid | 1 |

### Method of Preparation for Example 7

All ingredients excluding the hydrocortisone, fusidic acid and dry flo were melted down until molten, and the temperature of the bulk was then maintained at 60° C. The active ingredients and dry flo were carefully sheared into the bulk using a Silverson mixer. The bulk was solidified by exposing to low temperature, for example, 4° C. The solidified bulk was milled down and granulated also at low temperature, for example, 4° C.

Percentages are by weight based on the total weight of the combined ingredients.

1. A pharmaceutical composition for topical application to a mammalian patient, comprising a therapeutic agent and a pharmaceutically acceptable carrier, wherein the composition is solid at a temperature of about 25° C. or less, and, upon continuous contact with the skin of the patient, softens to a consistency to effect substantial application of the therapeutic agent to a desired skin area of the mammalian patient within a time period of less than 10 minutes.

2. A pharmaceutical composition as claimed in claim 1, wherein the composition is a solid unit dosage form.

3. A pharmaceutical composition as claimed in claim 1, wherein the composition has a surface area for contact with an area of skin not more than 400, 300, 250, 200, 150, 100, 50, 25, 20, 19, 18, 17, 16, 15, 14, 13, 12, 11, 10, 9, 8, 7, 6, 5, 4, 3, 2, 1, or 0.5 mm².

4. A pharmaceutical composition as claimed claim 1, wherein the therapeutic agent is a keratolytic agent, hormone modifier, steroid, antihistaminic, antibiotic, anti-fungal agent, anti-infection agent, antimicrobial agent, anti-viral agent, antiseptic, immunosuppressant agent, or an anti-ageing agent.

5. A pharmaceutical composition as claimed in claim 4, wherein the therapeutic agent is benzoyl peroxide, zinc, a vitamin D analogue, 5-fluoro uracil, salicylic acid, formaldehyde, glutaraldehyde, silver nitrate, imiquimod or podophyllum.

6. A pharmaceutical composition as claimed in claim 4, further comprising an antimicrobial agent, anti-inflammatory agent, anti-viral agent, antiseptic, anti-fungal agent, skin-cleaning agent, immunosuppressant agent and/or antibiotic.

7. A pharmaceutical composition as claimed in claim 1, further comprising one or more solvents.

8. A pharmaceutical composition as claimed in claim 7, wherein the solvent is ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil and/or silicone oil.

9. A pharmaceutical composition as claimed in claim 1, wherein the therapeutic agent is absorbed within a time period of less than about 10 minutes, less than about 5 minutes, less than about 3 minutes or less than about 1 minute following application to the skin.

10. A pharmaceutical composition as claimed in claim 1, wherein the composition comprises a compacted granulate of the therapeutic agent, and a pharmaceutically acceptable carrier medium, said compacted granulate having a softening point of not higher than skin temperature of a mammalian patient.

11. A pharmaceutical composition as claimed in claim 1, wherein the composition comprises a plurality of substantially solid particles comprising the therapeutic agent admixed with a pharmaceutically acceptable carrier medium, said particles having a softening point of not higher than skin temperature of a mammalian patient.

12. A pharmaceutical composition as claimed in claim 1, wherein the carrier one or more glycerides, cocoa butter, theobroma, one or more high molecular weight polyethylene glycol, one or more polyoxyethylene, lanolin and derivatives thereof, and one or more fatty acids, fatty alcohols and fatty esters, one or more organic oil, or one or more glycerides.

13. A pharmaceutical composition according to claim 1, wherein the composition is substantially free of preservatives.

14. A pharmaceutical composition as claimed in claim 1, further comprising one or more preservatives to prevent or reduce contamination of the composition during preparation.

15. A pharmaceutical composition as claimed in claim 1, substantially free of antioxidants.

16. A pharmaceutical composition as claimed in claim 1, wherein the composition comprises not less than about 60% by weight carrier based on the weight of the pharmaceutical composition, not less than about 80% by weight or not less than about 90% by weight.

17. A pharmaceutical composition as claimed in claim 1, wherein the composition further comprises an abrasive agent.

18. An applicator for applying a pharmaceutical composition as claimed in claim 1.

19. An applicator as claimed in claim 18, comprising a receiving means for receiving and carrying the pharmaceutical composition so that the composition may be applied directly to the skin and a grip for enabling a user to hold and manipulate the applicator.

20. A kit comprising at least one dose of a pharmaceutical composition for topical application to a mammalian patient, comprising a therapeutic agent and a pharmaceutically acceptable carrier, wherein the composition is solid at a temperature of about 25° C. or less, and, upon continuous contact with the skin of the patient, softens to a consistency to effect substantial application of the therapeutic agent to a desired skin area of the mammalian patient within a time period of less than 10 minutes, and an applicator.

21. A method of treating an animal or human body comprising topically applying a pharmaceutical composition as claimed in claim 1.

22. A pharmaceutical composition as claimed in claim 1, for treating acne, eczema, psoriases, urticaria, contact dermatitis, skin cancer, infections or other skin conditions.

23. An applicator as claimed in claim 18, for treating acne, eczema, psoriases, urticaria, contact dermatitis, skin cancer, infections or other skin conditions.

24. A pharmaceutical composition as claimed claim 1, wherein the composition is for administration to a mammalian patient in combination with an oral therapy.

25. A pharmaceutical composition as claimed in claim 24, wherein the oral therapy comprises one or more keratolytic agents, hormone modifiers, steroids, antihistaminics, antibiotics, anti-fungal agents, anti-infection agents, antimicrobial agents, anti-viral agents, antiseptics or immunosuppressant agents.

26. A pharmaceutical composition or product substantially as described in any one of the Examples.
